(19) United States
(12) Patent Application Publication
(10) Pub. No.: US 2002/0159960 A1
(43) Pub. Date: Oct. 31, 2002

(54) Method for improving the properties of transfer resistant lip compositions and related compositions and articles

(76) Inventors: Neil D. Scancarella, Wyckoff, NJ (US); Robert Walter Sandewicz, Monroe Township, NJ (US); Anjali Abhimanyu Patil, Westfield, NJ (US); Joseph Frank Calello, Bridgewater, NJ (US)

Correspondence Address: Julie Blackburn, Revlon Consumer Products Corporation, 625 Madison Avenue, New York, NY, 10022, US

(21) Appl. No.: 10/066,005

(22) Filed: Jan. 31, 2002

Related U.S. Application Data
(60) Provisional application No. 60/271,849, filed on Feb. 27, 2001.

(51) Int. Cl. A61K 7/025

(52) U.S. Cl. 424/64

(57) ABSTRACT

A method for improving the aesthetics of a pigmented, transfer resistant film on the lips, and a method for wetting said transfer resistant film, comprising coating said transfer resistant film with a non-reactive liquid wetting agent composition that serves to wet the transfer resistant film and improve the aesthetics; and a multipack cosmetic composition comprising at least two separate receptacles in a single stock keeping unit, the first receptacle containing a pigmented transfer resistant composition, and the second receptacle containing a non-reactive liquid wetting agent composition for said transfer resistant composition.

### RELATED APPLICATIONS

This application claims priority from provisional patent application serial No. 60/271,849 filed Feb. 27, 2001.

### TECHNICAL FIELD

The invention is in the field of lipstick compositions, methods for application, and related articles.

### BACKGROUND OF THE INVENTION

One of the common problems with lipstick is that it must be frequently reapplied as the color wears off very quickly, particularly when eating or drinking. A number of years ago, transfer resistant lipsticks were introduced. Such lipsticks were both long wearing in that they exhibited substantially improved adherence to the lips and at the same time did not transfer off when the lips came into contact/with objects such as eating utensils or cups. While transfer resistant lipsticks filled the need gap for longer wearing lipsticks, they created another need gap which was the desire for lip compositions that were more moisturizing to the lips. In particular, many consumers, particularly older women, perceived that transfer resistant lip formulas were more drying on the lips than their traditional lipsticks. While they liked the long wearing and transfer resistant benefits, they were not completely happy with the perceived lack of hydration. In general moisturizing lipsticks generally require the presence of emollient oils, which are precisely the ingredients that compromise long wear and transfer resistance. In other words, in order to optimize wear and provide transfer resistance, the oil level in the lipstick must be closely regulated because oils are marvelous solvents for the resins typically used to achieve these benefits. Solvating the resins, in turn, means that wear and transfer resistance is compromised. Thus, cosmetic companies are still trying to achieve optimal wear, transfer resistance, and hydration in one lip product.

One approach to this problem is set forth in U.S. Pat. Nos. 6,074,654; 6,071,503; and 6,019,962 assigned to Procter & Gamble. These patents describe a two pack system where the transfer resistant lipcolor is first applied to the lips and allowed to dry. Then, a second emollient layer in the form of a fatty oil based stick, is applied over the transfer resistant film. Patentees claim that this waxy overcoat composition improves the performance of the transfer resistant layer. However, the wear results for such a product appear to vary in consistency between individual so that wear and other aesthetics are somewhat unpredictable.

Another type of lipstick overcoat, disclosed in U.S. Pat. No. 5,750,094, is an example of an overcoat for a standard non-transfer resistant lipstick composition. This overcoat is designed to be compatible with the underlying color coat in that the silica and similar particles present combine with the oil found in the color coat and together, both compositions react to form a more transfer resistant lip coating. The difficulty with this approach is that the overcoat is both reactive and in some sense compatible with the color layer, and if used with a transfer resistant lipstick would interact with it and impact both wear and transfer resistance.

Accordingly, it has been discovered that achieving transfer resistance and improved aesthetics in a two pack system, requires that the transfer resistant and overcoat compositions exhibit a very specific and unique relationship. In particular, the transfer resistant color coating must adhere to the lips and provide long wear and resist transfer. The overcoat must be capable of application to the film, and while it should be non- reactive with the film, at the same time it should be capable of interaction with the film to a degree sufficient to adequately wet the film. It is only when the film is adequately wetted that the lipstick composition provides optimal feel, hydration, and comfort.

### SUMMARY OF THE INVENTION

The invention comprises a method for improving the aesthetics of a pigmented transfer resistant film on the lips comprising coating the transfer resistant film with a non-reactive wetting agent composition which may be in the form of a liquid where the wetting agent is the only ingredient present or the wetting agent is one of a mixture of ingredients present, or a solid or semi-solid composition wherein the liquid wetting agent comprises an ingredient, or mixture of wetting agent ingredients, in the composition. The wetting agent composition serves to wet the transfer resistant film, which in turn provides improved hydration, comfort, and wear to the composition on the lips.

The invention also comprises a method for wetting a pigmented transfer resistant film on the lips, comprising coating said transfer resistant film a non-reactive wetting agent composition that serves to wet the transfer resistant film.

The invention also comprises a multipack cosmetic composition for application to the lips comprising at least two separate receptacles in a single stock keeping unit, said first receptacle containing a pigmented transfer resistant composition, and said second receptacle containing a non-reactive wetting agent composition that serves to wet said transfer resistant film.

It is an object of the invention to provide a method for improving aesthetics such as feel, comfort, gloss, hydration, water resistance, etc., of transfer resistant films on the lips.

It is also an object of the invention to provide a method for wetting a pigmented, transfer resistant film on the lips, which in turn provides a cosmetic composition with improved aesthetics.

It is another object of the invention to provide a multipack cosmetic composition for application to the lips contained in at least two separate receptacles in a single stock keeping unit, said first receptacle containing a pigmented transfer resistant composition, and said second receptacle containing a non-reactive wetting agent composition that serves to wet said transfer resistant composition. The non-reactive wetting agent composition may be in the form of a liquid composition that is applied to the lips, or alternatively, it may be formulated into a semi-solid paste or solid stick composition.

It is another object of the invention to provide for a transfer resistant or long wearing cosmetic composition that provides good feel, comfort, water resistance, and lip hydration to the wearer.

It is another object of the invention to provide a multipack lip treatment system which provides color, hydration, and conditioning to the lips.

### BRIEF DESCRIPTION OF THE DRAWINGS

FIG. 1: depicts a cross section of a lip surface having a transfer resistant lip composition and wetting agent applied thereto, and illustrates how the wetting agent composition used in the method of the invention wets the transfer resistant film by providing a smooth, even coating on the internal and external surfaces of the transfer resistant film.

FIG. 2: depicts a cross section of a lip surface having a transfer resistant lip composition and wetting agent composition applied thereto, and illustrates a situation where the wetting agent composition does not wet the transfer resistant film, evidenced by beads on the internal and external surfaces of the film.

FIG. 3: is an illustration of one type of multipack cosmetic composition in accordance with the invention wherein the transfer resistant lip composition and the wetting agent composition are maintained in separate containers but in a single stock keeping unit.

FIG. 4: is an illustration of another type of multipack cosmetic composition in accordance with the invention wherein the transfer resistant lip composition and the wetting agent composition are kept in separate receptacles within a single container.

FIG. 5: illustrates another embodiment of the invention where the multipack cosmetic composition contains three separate receptacles in a single stock keeping unit for housing the transfer resistant lip composition, the wetting agent, and a remover composition.

FIG. 6: illustrates another embodiment of the invention where the multipack cosmetic composition contains three separate containers in a single stock keeping unit for housing the transfer resistant lip composition, the wetting agent composition, and a remover composition.

### DETAILED DESCRIPTION

I. The Methods of the Invention

The invention comprises a method for both wetting, and thereby improving the aesthetics of a pigmented, transfer resistant film on the lips, comprising coating said transfer resistant film with a non-reactive wetting agent composition that serves to wet the transfer resistant film. The non-reactive wetting agent composition may be a liquid wetting agent alone, or a liquid wetting agent composition that contains, in addition to the liquid wetting agent, other ingredients, and the final composition is a liquid, semi-solid, or solid at room temperature (25° C.). In addition the wetting agent composition may contain a liquid wetting agent in combination with other ingredients that provide a final composition that is in a semi-solid or solid form (such as a paste or stick).

1. Definitions.

The term “improving the aesthetics” means improving qualities such as feel on the lips, hydration, long-wear, gloss, transfer resistance, comfort of the color coat on the lips, water resistance, and general commercial attributes of the composition.

The term “wetting agent composition” means a composition that contains a liquid wetting agent alone, or in combination with other ingredients. The term “liquid” means that the wetting agent is a liquid at room temperature. The wetting agent composition (which contains the liquid wetting agent) may be in the form of a liquid, semi-solid, or solid.

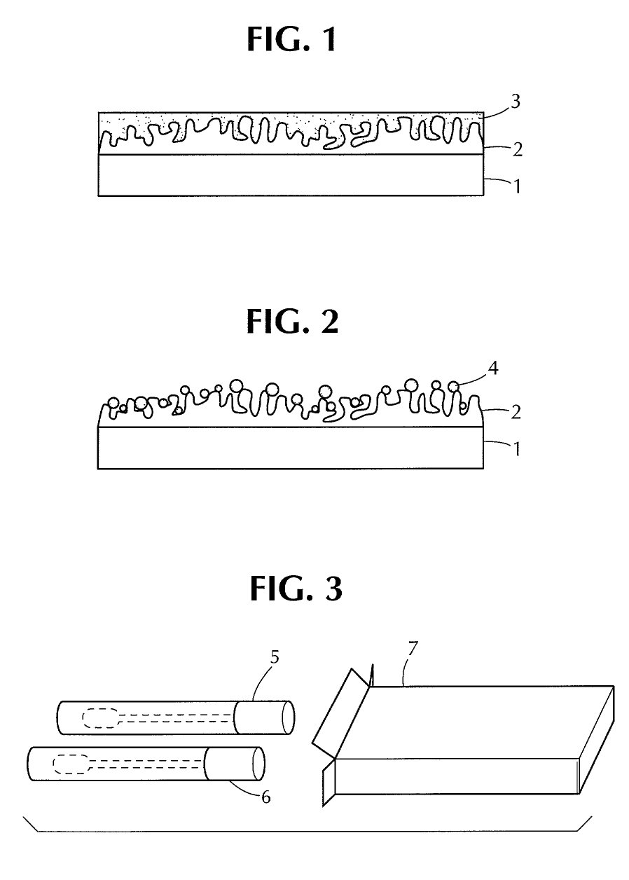

The term “wetting” means the ability of the transfer resistant film to be wet by the wetting agent composition. In accordance with this invention, the wetting agent composition is capable of adequately wetting the transfer resistant film. If the wetting agent composition is a fluid, the fluid flows smoothly over the internal and external film surfaces and any matrices that may exist within the film, and forms a smooth surface with no beading. In other words, if the wetting agent composition beads on the surfaces of the transfer resistant film, or within the matrices, it is not a suitable wetting agent for use in the claimed method. If the wetting agent composition is in the form of a semi-solid or solid composition, the liquid wetting agent present in the composition also serves to wet the transfer resistant film in the same manner, e.g. as the solid ingredients in the composition are softened by contact with the lips, the liquid wetting agent wets plasticizes the transfer resistant film. This is illustrated in FIGS. 1 and 2. FIG. 1 depicts a cross section of the lip surface 1, a transfer resistant film 2 applied thereon, and the wetting agent composition 3 which is applied to the film. The wetting agent composition has properly wetted the film, as is evident in the manner in which the wetting agent has evenly covered the film surfaces. FIG. 2 illustrates where the wetting agent composition does not adequately wet the transfer resistant film. The wetting agent composition has formed beads 4 on the transfer resistant film 2, and the film surfaces are not evenly covered.

In general wetting is more scientifically described by a measurement referred to as a “wetting angle” which describes the angle that exists between a specific liquid and a specific solid surface, and which gives an indication of the relative values of the forces of adhesion and cohesion that result in interfacial tension. In general, small wetting angles mean large wettability and large wetting angles mean small wettability. More specifically, if the wetting angle between a specific solid and a specific liquid is small, the liquid wets the solid to a more appreciable degree, and the adhesive forces of the liquid for the solid surface are greater than the internal cohesive forces of the liquid. Conversely, when the wetting angle between a specific solid and a specific liquid is relatively large, the liquid does not wet the solid to an appreciable degree, and the internal cohesive forces of the liquid are greater than the adhesive forces between the liquid and that particular solid surface. It is in the latter instance where beading is often seen. Preferably, in the method of the invention, when the wetting agent composition is applied to the transfer resistant film, the wetting angle between the wetting agent composition and the transfer resistant film surface is less than about 45 degrees.

The term “transfer resistant film” means a film that, when applied to lips, exhibits minimal transfer from the lips when the kiss test, as described in U.S. Pat. No. 5,505,937, which is hereby incorporated by reference, is performed. In particular, transfer resistance is assessed by applying the film to the lips and allowing it to dry for 5 minutes. Then, the lips are lightly touched to the back of the hand. If no color or a trace of color remains on the back of the hand, the film is considered transfer resistant.

The term “non-reactive” means that the wetting agent composition does not react with any of the ingredients in the transfer resistant film, by, for example, solvating or solubilizing any ingredient in the film, or otherwise reacting at any chemical level with any ingredient in the transfer resistant film.

The term “interactive” means that while the wetting agent composition is non-reactive with the transfer resistant film, it is still capable of physically interacting with the internal and external surfaces of the film by seeping into any matrices, and coating the surfaces of the transfer resistant film, and thereby wetting the film but at the same time being non-reactive and with the transfer resistant film.

B. The Lip Composition Used to Prepare the Transfer Resistant Film

The transfer resistant film that forms the color coating on the lips in the method of the invention is formed when a transfer resistant pigmented lip composition is applied to the lips. This composition generally comprises a volatile solvent in combination with pigments. The composition may additionally contain a polymeric film former.

1. Polymeric Film Formers

Generally, the lip composition comprises one or more polymeric film formers that are capable of forming a transfer resistant film on the lips when formulated into a pigment containing lip composition. Such compositions typically contain from about 0.1-50%, preferably 0.5-40%, more preferably 1-35% by weight of the total composition of polymeric film formers. Suitable polymeric film formers include synthetic polymers such as silicones and polymers made from ethylenically unsaturated monomers. Also suitable are natural polymers such as gums, resins, and similar materials derived from natural sources.

Suitable silicones include linear and cross-linked silicones that are resinous in character, e.g. which exhibit properties generally associated with resins such as film forming capability, substantivity, and occlusiveness. Examples of cross linked silicones suitable for use are siloxy silicate polymers having the following general formula:

[(RR′R″)₃SiO_{½}]ₓ[SiO₂]_{y}

wherein R, R′ and R″ are each independently a C₁₋₁₀ straight or branched chain alkyl or phenyl, and x and y are such that the ratio of (RR′R″)₃SiO_{½} units to SiO₂ units is 0.5 to 1 to 1.5 to 1. Preferably R, R′ and R″ are a C₁₋₆ alkyl, and more preferably are methyl and x and y are such that the ratio of (CH₃)₃SiO_{½} units to SiO₂ units is 0.75 to 1. Most preferred is this trimethylsiloxy silicate containing 2.4 to 2.9 weight percent hydroxyl groups which is formed by the reaction of the sodium salt of silicic acid, chlorotrimethylsilane, and isopropyl alcohol. The manufacture of trimethylsiloxy silicate is set forth in U.S. Pat. Nos. 2,676,182; 3,541,205; and 3,836,437, all of which are hereby incorporated by reference. Trimethylsiloxysilicate as described is available from GE Silicones under the tradename SR-1000, which is a solid particulate material. Also suitable is Dow Corning 749 which is a mixture of volatile cyclic silicone and trimethylsiloxysilicate.

Also suitable are linear, high molecular weight silicones that are semi-solids or solids at room temperature. Examples of such silicones include dimethicones having viscosities ranging from 500,000 to 10 million, dimethicone copolyols, or silicone waxes such as C₁₄₋₃₀ alkyl dimethicone waxes such as cetyl or stearyl dimethicone, or behenoxydimethicone. Preferably, the compositions are free of silicone gums.

Also suitable are silicone esters as disclosed in U.S. Pat. Nos. 4,725,658 and U.S. Pat. No. 5,334,737, which are hereby incorporated by reference. Such silicone esters comprise units of the general formula RₐR^{E}_{b}SiO_{[4-(a+b)/2]} or R¹³ₓR^{E}_{y}SiO_{½}, wherein R and R¹³ are each independently an organic radical such as alkyl, cycloalkyl, or aryl, or, for example, methyl, ethyl, propyl, hexyl, octyl, decyl, aryl, cyclohexyl, and the like, a is a number ranging from 0 to 3, b is a number ranging from 0 to 3, a+b is a number ranging from 1 to 3, x is a number from 0 to 3, y is a number from 0 to 3 and the sum of x+y is 3, and wherein R^{E} is a carboxylic ester containing radical. Preferred RE radicals are those wherein the ester group is formed of one or more fatty acid moieities (e.g. of about 2, often about 3 to 10 carbon atoms) and one or more aliphatic alcohol moieities (e.g. of about 10 to 30 carbon atoms). Examples of such acid moieties include those derived from branched-chain fatty acids such as isostearic, or straight chain fatty acids such as behenic. Examples of suitable alcohol moieties include those derived from monohydric or polyhydric alcohols, e.g. normal alkanols such as n-propanol and branched-chain etheralkanols such as (3,3,3-trimethylolpropoxy)propane. Preferably the ester subgroup (i.e. the carbonyloxy radical) will be linked to the silicon atom by a divalent aliphatic chain that is at least 2 or 3 carbon atoms in length, e.g. an alkylene group or a divalent alkyl ether group. Most preferably that chain will be part of the alcohol moiety, not the acid moiety. Such silicones may be liquids or solids at room temperature.

Other synthetic polymeric film formers that may be used for the lip composition are polymers made from various types of ethylenically unsaturated monomers, including esters of acrylic or methacrylic acid, or ethylene homo- or copolymers, or styrene homo- or copolymers. Such polymers may be homopolymer, copolymers, or graft or block copolymers. Examples of such polymeric film formers are disclosed in U.S. Pat. Nos. 6,143,283 and 6,153,206 which are hereby incorporated by reference in their entirety. U.S. Pat. No. 6,143,283 teaches acrylic polymers that are adhesives at room temperature. One type of such polymer comprises a backbone of vinyl, methacrylic, or acrylic monomers and pendant siloxane and fluorochemical groups, for example Poly(isobutyl methacrylate)-co-methyl FOSEA sold by 3M Company under the tradename SA 70-5 IBMMF. Also suitable are vinyl silicone graft or block copolymers, such as poly(dimethylsiloxane)-g-poly(isobutyl)methacrylate sold by 3M Company under the tradename VS 70. Also suitable are the methacrylate copolymers set forth in U.S. Pat. No. 6,153,206, which comprise uncrosslinked synthetic polymers comprising a first repeat unit selected from methacrylate ester monomers which, if polymerized, would yield a polymer having a glass transition temperature of −10 to 75° C., and a second repeat unit selected from methacrylate ester monomers which, if polymerized, would yield a polymer having a glass transition temperature of 76 to 120° C. Examples of such copolymers include those where the first repeat unit is selected from isobutyl methacrylate, ethyl methacrylate, hexyl methacrylate, and mixtures thereof; and the second repeat unit is selected from methyl methacrylate, isobornyl methacrylate or mixtures thereof.

Various types of natural film forming polymers may be used as well. Examples of such polymers include cellulosics and derivatives thereof, gums such as gum arabic, and resins obtained from trees and plants.

Preferably, the transfer resistant film in the method of the invention is formed with a lip composition containing a synthetic polymeric film former that is a silicone resin, in particular, trimethylsiloxysilicate.

2. Solvents

Typically the lip composition contains one or more ingredients that act as solvents for the polymeric film former. The polymeric film former generally remains dissolved in the solvents while the lip composition is stored in the container, but when it is applied to the lips the solvent tends to dissipate, at least in part, permitting the polymeric film former to form a transfer resistant film on the surface of the lips. Typical ranges of solvent are from about 1-98%, preferably 2-95%, more preferably 15-85% by weight of the total composition. Suitable solvents may be volatile or non-volatile and are liquids at room temperature. The term “volatile” means that the oil has a measurable vapor pressure, or a vapor pressure of at least 2 mm. of mercury at 20° C. The term “nonvolatile” means that the oil has a vapor pressure of less than 2 mm. of mercury at 20° C. Suitable volatile solvents generally have a viscosity of 0.5 to 10 centistokes at 25° C. Suitable volatile oils include linear silicones, cyclic silicones, paraffinic hydrocarbons, or mixtures thereof

Cyclic silicones are of the general formula:

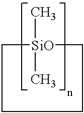

where n=3-7.

Linear volatile silicones in accordance with the invention have the general formula:

(CH₃)₃Si—O—[Si(CH₃)₂—O]ₙ—Si(CH₃)₃

where n=0-6, preferably 0-5.

Linear and cyclic volatile silicones are available from various commercial sources including Dow Corning Corporation and General Electric. The Dow Corning volatile silicones are sold under the tradenames Dow Corning 244, 245, 344, and 200 fluids. These fluids comprise octamethylcyclotetrasiloxane, decamethylcyclopentasiloxane, hexamethyldisiloxane, and mixtures thereof.

Also suitable as the volatile oils are various straight or branched chain paraffinic hydrocarbons having 5 to 40 carbon atoms, more preferably 8-20 carbon atoms. Suitable hydrocarbons include pentane, hexane, heptane, decane, dodecane, tetradecane, tridecane, and C₈₋₂₀ isoparaffins as disclosed in U.S. Pat. Nos. 3,439,088 and 3,818,105, both of which are hereby incorporated by reference. Preferred volatile paraffinic hydrocarbons have a molecular weight of 70-225, preferably 160 to 190 and a boiling point range of 30 to 320, preferably 60-260 degrees C., and a viscosity of less than 10 cs. at 25 degrees C. Such paraffinic hydrocarbons are available from EXXON under the ISOPARS trademark, and from the Permethyl Corporation. Suitable C₁₂ isoparaffins are manufactured by Permethyl Corporation under the tradename Permethyl 99A. Another C₁₂ isoparaffin (isododecane) is distributed by Presperse under the tradename Permethyl 99A. Various C₁₆ isoparaffins commercially available, such as isohexadecane (having the tradename Permethyl R), are also suitable. A particularly preferred solvent for use in the transfer resistant lipstick composition is isododecane.

A wide variety of nonvolatile oils are also suitable for use as solvents for the film forming polymer in the composition. The nonvolatile oils generally have a viscosity of greater than 10 centipoise at 25° C., and may range in viscosity up to 500,000 centipoise at 25° C. Particularly preferred are nonvolatile organic oils or silicone fluids, both water soluble and water insoluble. Preferred silicones have a viscosity of 10 to 100,000 centistokes, preferably 20 to 50,000 centistokes at 25° C. Suitable water insoluble silicones include amodimethicone, dimethicone, dimethicone copolyol, phenyl trimethicone, diphenyl methylsiloxane, and mixtures thereof Also suitable are water soluble silicones such as dimethicone copolyol, dimethiconol, and the like. Such silicones are available from Dow Coming as the 3225C formulation aid, Dow 190 and 193 fluids, or similar products marketed by Goldschmidt under the ABIL tradename.

Also suitable as the nonvolatile oil are fluorinated silicones such as trimethylsilyl endcapped fluorosilicone oil, polytrifluoropropylmethylsiloxanes, and similar silicones such as those disclosed in U.S. Pat. No. 5,118,496 which is hereby incorporated by reference.

Also suitable for the nonvolatile oils are organic oils such as esters, hydrocarbons, fatty alcohols, and the like.

Suitable esters are mono-, di-, and triesters. Monoesters are defined as esters formed by the reaction of a monocarboxylic acid having the formula R—COOH, wherein R is a straight or branched chain saturated or unsaturated alkyl having 2 to 30 carbon atoms, or phenyl; and an alcohol having the formula R—OH wherein R is a straight or branched chain saturated or unsaturated alkyl having 2-30 carbon atoms, or phenyl. Both the alcohol and the acid may be substituted with one or more hydroxyl groups. Either one or both of the acid or alcohol may be a “fatty” acid or alcohol, ie. may have from about 6 to 22 carbon atoms. Examples of monoester oils include hexyldecyl benzoate, hexyl laurate, hexadecyl isostearate, hexydecyl laurate, hexyldecyl octanoate, hexyldecyl oleate, hexyldecyl palmitate, hexyldecyl stearate, hexyldodecyl salicylate, hexyl isostearate, butyl acetate, butyl isostearate, butyl oleate, butyl octyl oleate, cetyl palmitate, ceyl octanoate, cetyl laurate, cetyl lactate, cetyl isononanoate, cetyl stearate, stearyl lactate, stearyl octanoate, stearyl heptanoate, stearyl stearate, and so on. It is understood that in the above nomenclature, the first term indicates the alcohol and the second term indicates the acid in the reaction, i.e. stearyl octanoate is the reaction product of stearyl alcohol and octanoic acid.

Suitable diesters are generally formed by the reaction of a dicarboxylic acid and an aliphatic or aromatic alcohol. The dicarboxylic acid may contain from 2 to 30 carbon atoms, and may be in the straight or branched chain, saturated or unsaturated form. The dicarboxylic acid may be subsituted with one or more hydroxyl group. The aliphatic or aromatic alcohol may also contain 2 to 30 carbon atoms, and may be in the straight or branched chain, saturated, or unsaturated form. The aliphatic or aromatic alcohol may be substituted with one or more substitutents such as hydroxyl. Preferably, one or more of the acid or alcohol is a fatty acid or alcohol, i.e. contains 14-22 carbon atoms. The dicarboxylic acid may also be an alpha hydroxy acid. Examples of diester oils that may be used in the compositions of the invention include diisostearyl malate (the reaction product of isostearic alcohol and malic acid), neopentyl glycol dioctanoate (the reaction product of neopentyl glycol and 2-ethyl hexanoic acid), dibutyl sebacate (reaction product of butyl alcohol and sebacic acid), di-C₁₂₋₁₃ alkyl malate (reaction product of C₁₂₋₁₃ alcohol and malic acid), dicetearyl dimer dilinoleate (reaction product of cetearyl alcohol and adipic acid), dicetyl adipate (reaction product of cetyl acohol and adipic acid), diisocetyl adipate (reaction product of hexadecyl alcohol and adipic acid), diisononyl adipate (reaction product of isononyl alcohol and adipic acid), diisostearyl dimer dilinoleate (reaction product of isostearyl alcohol and dilinoleic acid), disostearyl fumarate (reaction product of isostearyl alcohol and fumaric acid), and so on.

Suitable triesters comprise the reaction product of a tricarboxylic acid and an aliphatic or aromatic alcohol. As with the mono- and diesters mentioned above, the acid and alcohol contain 2 to 30 carbon atoms, and may be saturated or unsatured, straight or branched chain, and may be substituted with one or more hydroxyl groups. Preferably, one or more of the acid or alcohol is a fatty acid or alcohol containing 14 to 22 carbon atoms. Examples of triesters include triarachidin (reaction product of glycerin and arachidic acid), tributyl citrate (reaction product of butyl alcohol and citric acid), tri C12-13 alkyl citrate (reaction product of C12-13 alcohol and citric acid), tricaprylin (reaction product of glycerin and caprylic acid), tricaprylyl citrate (reaction product of capryl alcohol and citric acid), tridecyl behenate (reaction product of tridecyl alcohol and behenic acid), trioctyldodecyl citrate (reaction product of octyldodecyl alcohol and citric acid), tridecyl behenate (reaction product of tridecyl alcohol and behenic acid), tridecyl cocoate (reaction product of tridecyl alcohol and coconut acid), tridecyl isononanoate (reaction product of tridecyl alcohol and isononanoate), and so on.

Suitable nonvolatile hydrocarbon oils include isoparaffins and olefins having greater than 20 carbon atoms. Examples of such hydrocarbon oils include C₂₄₋₂₈ olefins, C₃₀₋₄₅ olefins, C₂₀₋₄₀ isoparaffins, hydrogenated polyisobutene, mineral oil, pentahydrosqualene, squalene, squalane, and mixtures thereof Straight or branched chain fatty alcohols are also suitable. Generally, such alcohols have the formula R—OH wherein R is a straight or branched chain saturated or unsaturated alkyl having 6-30 carbon atoms, are also suitable oils. Such fatty alcohols include cetyl alcohol, stearyl alcohol, cetearyl alcohol, and the like.

3. Pigments

The lip composition used to form the transfer resistant film in the method of the invention also comprises pigments. The term “pigment” when used herein means both pigments and powder materials traditionally used for muting color or providing bulk.

Typically such transfer resistant compositions contain 0.5-50%, preferably 7-45%, more preferably 10-40%, by weight of the total composition, of pigments having a particle size of 0.02 to 100, preferably 0.5 to 100, microns. Examples of pigments include bismuth oxychloride, titanated mica, fumed silica, spherical silica, polymethylmethacrylate, polyethylene, polypropylene, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, or mixtures thereof The above mentioned powders may be surface treated with lecithin, amino acids, mineral oil, silicone oil or various other agents either alone or in combination, which coat the powder surface and render the particles more lipophilic in nature.

Various organic and inorganic pigments that may be used include include azo, indigoid, triphenylmethane, anthraquinone, and xanthine dyes which are designated as D&C and FD&C blues, browns, greens, oranges, reds, yellows, etc. Organic pigments generally consist of insoluble metallic salts of certified color additives, referred to as the Lakes, in particular the Lakes of D&C and FD&C colors. Inorganic pigments include iron oxides, ultramarines, chromium, chromium hydroxide colors, and mixtures thereof

4. Thickening or Gelling Agents

The lip compositions used to form the transfer resistant film may also comprise one or more thickening or gelling agents which impart increased viscosity to the final composition. Typically the compositions comprise 0.1-40%, more preferably 1-15%, more preferably 1-10% by weight of the total composition of such agents. Examples of thickening agents include natural or synthetic waxes, natural or synthetic metal silicates, and derivatives thereof Examples of synthetic silicate gelling agents are disclosed in U.S. Pat. No. 3,586,478, which is hereby incorporated by reference. Natural silicates are typically extracted from quartz or dolomite and are often referred to as “clay”. Particularly preferred are natural silicates such as montmorillonite minerals and quaternized derviatives thereof The term “quaternized derivatives” means that the montmorillonite minerals are reacted with one or more quaternary ammonium compounds such as quaternium-18, quaternium-16, etc. Examples of suitable waxes include those set forth in U.S. Pat. No. 5,725,845 which is hereby incorporated by reference. Particularly preferred are quaternized montmorillonite minerals including quaternium-18 hectorite. or quaternium-18 bentonite.

5. Other Ingredients

The lip compositions used to form the transfer resistant film may also contain other ingredients such as preservatives, antioxidants, extracts, and so on, so long as these ingredients do not impair the transfer resistant characteristics of the composition.

The lip compositions may be in the form of liquids, solids, or semi-solids. Preferably, the lip composition that forms the transfer resistant film is in the liquid or semi-solid form.

C. Application of the Transfer Resistant Film

The lip composition as described above is applied to the lips in the usual manner. The manner of application will depend on whether the lip composition is in the form of a solid, liquid, or semi-solid. If in the solid form, the lip composition is probably in the form of a stick, or solidified into a pan with an accompanying applicator. If in the semi-solid or liquid form, the lip composition is probably is dispensed from a vial or similar container using a doe foot applicator or brush. After the lip composition is applied to the lips it is allowed to dry for the appropriate period of time. Drying time will depend on the type of polymeric film former and other ingredients in the composition. Generally, a drying time of ½ minute to 10 minutes is suitable to permit the transfer resistant layer to adequately set on the lips. While the best results are obtained if the lip composition is allowed to dry completely prior to application of the wetting agent composition, it is not necessary. In most cases, the transfer resistant layer that is formed on the lips does not feel like normal lipstick. The film may feel tight and dry depending on the polymeric film former used in the transfer resistant composition.

D. The Wetting Agent Composition

The wetting agent composition that is applied to the transfer resistant film in the method of the invention is a liquid wetting agent ingredient or combination of ingredients including the wetting agent that are non-reactive with the transfer resistant film but at the same time capable of interacting with the internal and external surfaces and matrices of the film by seeping into spaces and surfaces in a smooth, even fashion, to a degree sufficient to wet the transfer resistant film. A variety of wetting agents may be used in the composition so long as they fulfill the criteria described herein.

1. High Viscosity Natural Oils

Particularly suitable as the wetting agent are high viscosity natural oils derived from plant or animal sources. High viscosity generally means that while such oils are liquids at room temperature they have a viscosity greater than 100 centipoise at room temperature and less than about 1,000,000. More preferably, the oils have viscosities ranging from about 300 to 800,000 centipoise at 25° C., most preferably 300 to 100,000 centipoise. Preferably, such oils also have a refractive index ranging from about 1.0 to 1.5. Particularly preferred is lanolin oil and derivatives thereof Lanolin oil is a refined derivative from the unctuous fat-like sebaceous secretion from sheep.

2. Hydrocarbons

Also suitable are polymeric hydrocarbons preferably having number average molecular weights greater than about 650. Examples of such polymers include polybutenes such as Indopol H-100, Indopol H-50, and Parapol 700, manufactured by Amoco. Also suitable are polymeric alpha olefins obtained by the polymerization of alpha olefins. Particularly preferred are Synton PAO 100 manufactured by Uniroyal Chemical, Puresyn 150 and Puresyn 100, manufactured by ExxonMobil Chemical. Puresyn 150, 100, and 300 are referred to as hydrogenated polydecene.

3. Surfactants

Also suitable as the liquid wetting agent are surfactants that are more hydrophilic than hydrophobic in character. Particularly suitable are certain types of alkoxylated alcohols which are ethers formed by the reaction of an alkylene oxide such as ethylene or propylene oxide with a polyol. Preferably, the alkylene oxide is propylene oxide and the polyol is a carbohydrate such as glucose, methyl glucose, fructose, xylose, arabinose, and so on. Particularly preferred is an alkoxylated alcohol formed by the reaction of propylene oxide and methyl glucose, wherein the number of repeating propylene oxide units ranges from about 2 to 100. Most preferred is PPG-10 methyl glucose ether, sold by Amerchol under the tradename Glucam P-10.

4. Glyceryl Esters

Also suitable for use as the wetting agent are glyceryl esters formed by the reaction of glycerin and C₁₋₁₀ carboxylic acids. Such esters may be mono-, di- or triesters. Preferably, the glyceryl esters are triesters formed by the reaction of glycerin with a C₁₋₄ straight chain carboxylic acids. Particularly preferred is triacetin, which is the triester of glycerin and acetic acid.

5. Dihydric or Polyhydric Alcohols

Also suitable are various types of aliphatic dihydric or polyhydric alcohols, including glycerin, propylene glycol, butylene glycol, and the like.

6. Water Soluble Polymers

Also suitable are certain types of water soluble homo- or copolymers made from ethylenically unsaturated monomers either alone or copolymerized with various types of organic compounds. Examples of such polymers include vinylpyrrolidones, copolymers of vinyl pyrrolidone and vinyl acetate (PVP/VA copolymer), and the like. Also suitable are such polymers where the substituent groups have been reacted with various organic compounds, for example the esterification of polymethacrylic acid with one or more aliphatic or aromatic alcohols. Particularly preferred are homo- or copolymers containing acrylic acid or methacrylic acid monomers wherein the acid monomers have been esterified with mono-, di-, or polyhydric alcohols. One example of such a polymer is glyceryl polymethacrylate which is the ester of the polyhydric alcohol glycerin and polymethacrylic acid. Another example of such a polymer is PVP/carbamyl polyglycol ester.

7. Perfluoropolyethers

Also suitable for use as the wetting agent are various types of perfluoropolyethers as described in U.S. Pat. No. 3,665,041, which is hereby incorporated by reference. Such perfluoropolyethers have the general formula:

X—O(C₃F₆—O)ₚ(CF₂—O)_{q}(C₂F₄—O)ᵣ—Y

in which —C₃F₆— and —C₂F₄— are perfluoroalkylene groups having the structure:

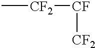

and —CF₂—CF₂, respectively, and p, q, and r are average indices and only p and r can be zero and p, q, and r together provide a polymer having a molecular weight ranging from 100 to 100,000; and X and Y are selected from —CF₂—, —C₂F₅—, and —C₃F₇—, and

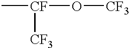

Particularly preferred is a polymer wherein p is zero, and q and r are such that the final polymer has a molecular weight ranging from about 500 to 50,000; and X and Y are CF₃. Such compounds are manufactured by Ausimont under the trademark Fomblin, such as Fomblin HC/R, HC/01, HC/02, HC/03 and the like. Particularly preferred is Fomblin HC/R which is a clear, colorless liquid having a refractive index of 1.299 to 1.305, a viscosity of 1050 to 1550 centistokes at 20° C., having the INCI name polyperfluoropolymethylisopropyl ether.

Also suitable is a perfluoropolyether manufactured by Advanced Polymer Systems which is sold under the tradename Afflunox oil. This oil has repeating units of the following general formula:

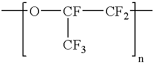

wherein the degree of polymerization varies from about 8 to 100.

The wetting agent may consist of only one ingredient or a mixture of ingredients in the form of a composition, provided the wetting agent compound or composition contains the specifications mentioned herein and remains non-reactive, yet interactive with the transfer resistant film.

Preferably, the wetting agent has a refractive index ranging from 1.0 to 1.5.

E. The Wetting Agent Composition

If desired the liquid wetting agent may be incorporated into a liquid, semi-solid, or solid composition wherein additional ingredients may be present, such as waxes, oils, humectants, colorants, preservatives, antioxidants, and so on. Suitable amounts of wax range from about 0.01-40% by weight of the total composition. Such waxes may be animal, vegetable, mineral, or synthetic waxes, and will generally have a melting point ranging from about 30 to 150° C. Preferred waxes include synthetic wax, polyethylene, microcrystalline waxes, mineral waxes, silicone waxes, and so on, as disclosed in U.S. Pat. No. 5,505,937, which is hereby incorporated by reference in its entirety. Suitable ranges of oils (other than the liquid wetting agent) are about 0.1-50% by weight of the total composition and include volatile and non-volatile silicone oils, esters, triglycerides, and so on. Suitable ranges of humectants are from about 0.1-20% by weight of the total composition and include glycerin, polyols, glyceryl esters, and so on.

F. Application of the Wetting Agent

The transfer resistant lip composition is first applied to the lips. Preferably, this film is permitted to dry for at least 30 seconds to 5 minutes although this is not completely necessary. In fact, in some cases, application of the wetting agent to the transfer resistant film without permitting the transfer resistant film to dry first will provide wetting that is equivalent to that achieved when the film is permitted to dry. The wetting agent composition does not interfere with the transfer resistant character of the lip composition because it is selected so as to be non-reactive and with the transfer resistant film. However, the wetting agent composition will cause improved wetting of the film, which in turn improves the aesthetics. The improved aesthetics include hydration, comfort, feel, gloss. The wetting agent composition will wear away during the day or when eating or drinking, and may simply be reapplied at desired intervals in order to maintain the transfer resistant film in a comfortable state. The transfer resistant film will remain on the lips for an extended period of time, possibly even until removed with a chemical remover. If the transfer resistant film is particularly adherent, certain chemical removers may be used to remove it. FIG. 1 depicts how the wetting agent composition should wet the transfer resistant film in accordance with the method of the invention. FIG. 2 shows wetting that is inadequate. In particular, the wetting agent composition has formed beads on the film surface and within the matrices.

II. The Multipack Cosmetic Composition

The invention also comprises a multipack cosmetic composition for application to the lips comprising at least two separate receptacles in a single stock keeping unit, said first receptacle containing a pigmented transfer resistant composition, and said second receptacle containing an non-reactive wetting agent composition for said transfer resistant composition.

The term “multipack cosmetic composition” means a cosmetic composition that is applied in at least two separate steps requiring at least two separate compositions housed in separate containers or separate receptacles.

The term “single stock keeping unit” means that the multipack is sold as a single item with the different cosmetic components in separate receptacles. The receptacles may be in one single container or two or more separate containers sold as a single stock keeping unit. If the latter, preferably, the multipack is scanned under a single UPC code.

The containers may be made of suitable materials generally used for this purpose including plastics such as ABS, styrene, polyethylene and so on. Typically such containers are cylindrical vials with screw cap closures. Generally, the applicators used to apply the products are affixed to the inner top surface of the screw cap closures.

FIG. 3 depicts one type of multipack cosmetic composition in accordance with the invention. One receptacle is a separate container 5 which contains the transfer resistant lipstick composition and the other receptacle is a separate container 6 that contains the wetting agent composition. The containers are generally sold together in a single stock keeping unit in the form of a blister pack 8 (see FIG. 5) or in a box 7, or other similar unit.

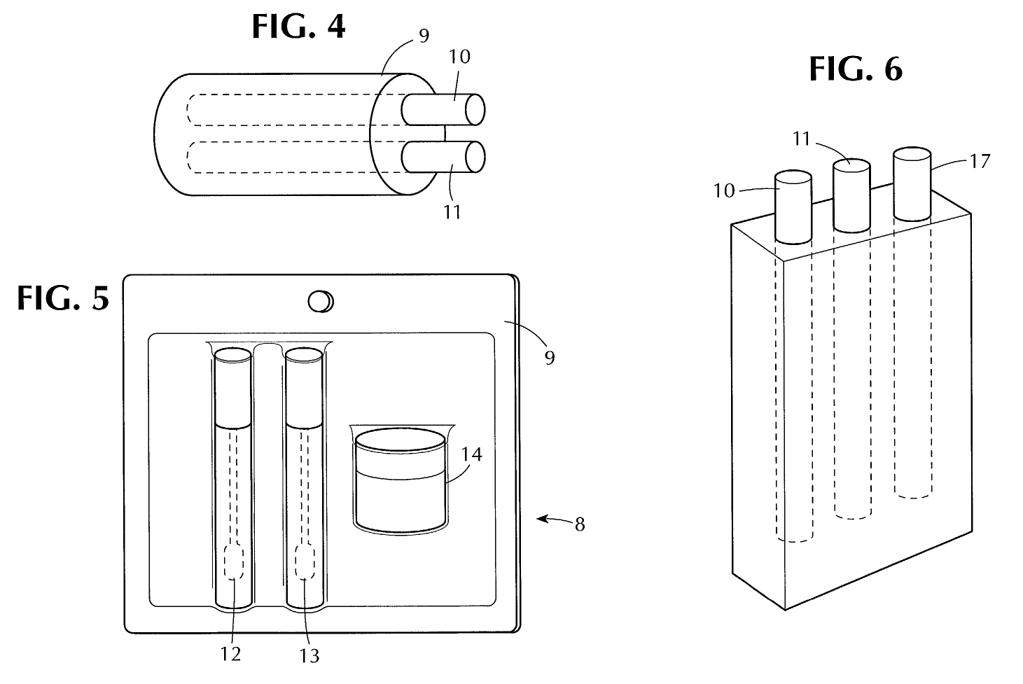

FIG. 4 illustrates another multipack cosmetic composition in accordance with the invention. The multipack cosmetic composition is housed in a single container 9 that contains two separate receptacles 10 and 11. One receptacle contains the transfer resistant lip product and the other receptacle contains the wetting agent composition.

FIG. 5 illustrates another embodiment of the invention showing a multipack cosmetic composition comprised of three containers for housing the transfer resistant lip product 12, the wetting agent 13, and a remover composition 15. The remover composition 15 is used to remove the lipstick from the lips. The three containers may be sold in a box (see FIG. 3) or in a blister pack 8 where the three containers are affixed to the blister card 9 by means of vacuum formed plastic 10 in the usual fashion.

FIG. 6 illustrates yet another embodiment of the invention where the transfer resistant lipstick composition 14, the wetting agent composition 15, and the remover composition 16 are housed in a single container having three separate receptacles.

The multipack cosmetic composition of the invention enables the consumer to obtain a long wearing, transfer resistant lip finish which exhibits all of the aesthetics mentioned. This attributes are achieved because the film is adequately wetted by the wetting agent composition, and at the same time the wetting agent composition is non-reactive and with the transfer resistant film so it does not solvate or otherwise chemically react with the film. The lip color film will remain on the lips for extended periods of time, and will generally not transfer to eating utensils, glassware, and the like upon contact. The wetting agent composition will eventually wear off the lips, but is reapplied at desired intervals to maintain the aesthetic benefits. The wetting agent composition provides a protective film over the transfer resistant film in addition to the other benefits mentioned.

The invention will be further described in connection with the following examples, which are set forth for the purposes of illustration only.

### EXAMPLE 1

A transfer resistant color coat was prepared according to the following formula:

| | w/w % |
|---|---|
| Trimethylsiloxysilicate/isododecane (50/50) | 31.5 |
| Quaternium-18 hectorite/isododecane/ | 20.00 |
| propylene carbonate (87/10/3) | |
| Isododecane | 27.50 |
| FD&C Red #7 in isododecane (28%) | 10.00 |
| Mica | 7.00 |
| Synthetic wax | 2.50 |
| Fluorosilicone | 1.50 |

All ingredients except the fluorosilicone were combined and heated to a temperature of 85 to 90° C. and mixed well. The mixture was cooled to room temperature. The fluorosilicone was added and the mixture heated again to a temperature of 85 to 90° C. until the fluorosilicone had become well mixed. The mixture was cooled to room temperature. The composition, a semi-solid paste, was put into a pot.

### EXAMPLE 2

The color coat of Example 2 was tested with the following overcoats. The color coat was applied to the lips and allowed to dry. The overcoat was applied and allowed to set for 15 to 30 minutes. The overcoat was then wiped with a tissue. The amount of color that transferred to the tissue was evaluated on a 0 to 3 scale, with the—sign in front of the number indicating that the overcoat performed slightly worse than the number following it.

| Rating | |
|---|---|
| 0 | No transfer (wetting agent non-reactive and interactive with |
| | transfer resistant film) |
| 1 | Some color transfer (wetting agent reactive and compatible with |
| | transfer resistant film) |
| 2 | Substantial amount of color transfer (wetting agent reactive and |
| | compatible with transfer resistant film) |
| 3 | Basecoat removed (wetting agent reactive and compatible with |
| | transfer resistant film) |

| | |
|---|---|
| OVERCOAT | RATING |

OILS

| | |
|---|---|
| Ricinus Communis seed oil | 1 |
| Lanolin oil | −1 |
| Apricot kernel oil | 3 |
| Sesame oil | 3 |
| Sweet almond oil | 3 |
| Synthetic jojoba oil | 3 |
| Tricaprylin this is an ester liquid | 3 |

HYDROCARBONS

| | |
|---|---|
| Polybutene (Indopol H-100 Mn* 940) | 1 |
| Polybutene (Indopol H-15 Mn 600) | 3 |
| Polybutene (Indopol H-50 Mn 815) | 1 |
| Polybutene (Indopol L-14 Mn 370 and L-50 Mn 455) | 3 |
| Polybutene (Parapol 450 Mn 420) | 3 |
| Polybutene (Parapol 700 Mn 700) | −1 |
| Polyalphaolefin (Synton PAO 100) | 1 |
| Hydrogenated polyisobutene | 3 |
| Decene/butene copolymer | 3 |

POLYURETHANES

| | |
|---|---|
| PPG 51/SMDI copolymer (Polyprepolymer 14) | 2 |
| PEG-2 soyamine/IPDI copolymer (Polyderm PPI-SA) | 3 |
| PEG 15 cocamine/IPDI copolymer (Polyderm PPI-CO-15) | 2 |
| Glycereth-7-diglycerol PEG-15 cocamine/IPDI copolymer | 2 |
| (Polyderm PPI-G7/CA) | |
| Polydiethylene glycol adipate/IPDI copolymer | 2 |
| (Polyderm PPI-PE) | |
| Castor oil/IPDI copolymer (Polyderm PPI-CO) | 2 |

SURFACTANTS

| | |
|---|---|
| PEG-25 hydrogenated castor oil | 2 |
| PPG-24 glycereth-24 | 3 |
| PEG-40 sorbitan peroleate | 3 |
| PEG-7 glyceryl cocoate | 3 |
| Polysorbate 80 | 2 |
| PPG-stearyl alcohol | 3 |
| PPG-10 methyl glucose ether | 1 |

ESTERS

| | |
|---|---|
| Triacetin | −1 |
| Acetyl tributyl citrate | 3 |
| PPG-2 dibenzoate | 2 |
| Isononyl isononanaoate | 3 |
| Trioctyl dodecyl citrate | 3 |
| Isostearyl hydroxy stearate | 3 |
| Diisostearyl maleate | 3 |
| Pentaerythritol tetraoctanoate | 3 |
| Triisostearyl citrate | 2 |
| Diisostearyl adipate | 3 |
| Diisopropyl sebacate | 3 |

MISCELLANEOUS

| | |
|---|---|
| Phytantriol | 3 |
| C10-30 cholesterol/lanosterol esters | 2 |
| Abietic alcohol | 2 |
| Octyl dodecanol | 3 |
| Propylene carbonate | 2 |
| PVP/hexadecene copolymer | 2 |
| Dimethicone fluid (350 cs) | 3 |
| Dimethicone copolyol (Dow Coming 1248 fluid) | 3 |
| Dimethicone gum in cyclomethicone (Dow Corning 1401 | 3 |
| fluid) | |
| Fluorosilicone (trifluoroethyl C₁₋₄ dimethicone) | 3 |
| Glycerine | 1 |
| Propylene glycol | 1 |
| Butylene glycol | 1 |
| Polyvinylpyrrolidone | 1 |
| PVP/VA copolymer | 1 |
| Glyceryl polymethacrylate | 1 |
| PVP/carbamyl polyglycol ester | 1 |
| Polyperfluoropolymethylisopropyl ether (Fomblin HC/R) | 1 |

*Number average molecular weight.

### EXAMPLE 3

Transfer resistant lipcolor formulas were made as follows:

w/w %

| | 1 | 2 |
|---|---|---|
| Polymer solution¹ | 92.0 | 45.00 |
| PPG-2 dibenzoate | 3.75 | — |
| D&C Red #7 in lanolin oil² | 4.25 | 10.00 |
| Dimethicone (0.65 cs.) | — | 30.00 |
| VS-70³ | — | 15.00 |

¹A solution of 50% by weight of a copolymer comprised of isobutyl methacrylate and isobornyl methacrylate and 50% by weight of isododecane.
²33% by weight D&C Red #7 and 67% by weight lanolin oil.
³A solution of 45% by weight poly(dimethylsiloxane)-g-poly(isobutyl methacrylate) and 55% isododecane, 3M Company.

The color coats 1 and 2, above, were tested with the following overcoats. The color coat was applied to the lips and allowed to dry. The overcoat was applied and allowed to set for 15 to 30 minutes. The overcoat was then wiped with a tissue. The amount of color that transferred to the tissue was evaluated on the same 0 to 3 scale as set forth in Example 2.

| | RATING - | RATING - |
|---|---|---|
| OVERCOAT | Formula 1 | Formula 2 |
| Polyperfluoropolymethyliso | 0 | 0 |
| propyl ether (Fomblin | | |
| HC/R) | | |
| Ricinus Communis seed oil | 3 | 3 |
| Polyalphaolefin (Synton | −0 | 0 |
| PAO 100) | | |
| Diisostearyl malate | 0 | 1 |
| Sweet almond oil | 3 | 3 |
| PPG-10 methyl glucose | 0 | 0 |
| ether | | |
| Polybutene (Indopol L-14) | 2 | 3 |
| Mn 370 | | |
| Polybutene (Indopol H50) | 1 | 0 |
| Mn 815 | | |
| Dimethicone 350 Cs | −0 | 0 |
| Fluorosilicone | 0 | 0 |
| Dimethicone copolyol | 0 | 0 |
| (Dow Corning 1248 Fluid) | | |
| Dimethicone gum in | −2 | 0 |
| cyclomethicone (Dow | | |
| Corning 1401 Fluid) | | |

### EXAMPLE 4

A wetting agent composition in the solid form in accordance with the invention was made as follows:

| | w/w % |
|---|---|
| Polyethylene | 10.25 |
| Cyclomethicone | 25.50 |
| Polyalphaolefin (Puresyn 150)* | 39.85 |
| Polyalphaolefin (Puresyn 100)** | 24.00 |
| Triclosan | 0.10 |
| Benzoic acid | 0.20 |
| Butylated hydroxy toluene | 0.1 |

*hydrogenated polydecene, M.W. 3,500
**hydrogenated polydecene MW 2,900

The composition was prepared by combining the ingredients with sufficient warming, mixing well, and pouring into stick molds and allowing to cool.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. A method for improving the aesthetics of a pigmented, transfer resistant film on the lips, comprising coating said transfer resistant film with a non-reactive wetting agent composition that wets the transfer resistant film.

2. The method of claim 1 wherein the aesthetics improved are feel, comfort, transfer resistance, hydration, long wear, water resistance, and gloss.

3. The method of claim 1 wherein the pigmented, transfer resistant film is a resinous, pigmented film.

4. The method of claim 3 wherein the resinous pigmented film comprises a silicone resin or acrylic polymer.

5. The method of claim 4 wherein the resinous pigmented film comprises a silicone resin or acrylic polymer in combination with a volatile solvent.

6. The method of claim 5 wherein the resinous pigmented film comprises a silicone resin and the volatile solvent comprises a volatile paraffinic hydrocarbon or a volatile silicone.

7. The method of claim 6 wherein the resinous, pigmented film comprises a silicone resin in combination with one or more nonvolatile silicones, esters, or fatty alcohols.

8. The method of claim 1 wherein the wetting agent composition comprises a high viscosity oil, hydrocarbon, surfactant, glyceryl ester, di- or polyhydric alcohol, water soluble polymer, polyalphaolefin, or perfluoropolyether.

9. The method of claim 8 wherein the wetting agent composition comprises a perfluoropolyether or a polyalphaolefin.

10. The method of claim 1 wherein the wetting agent composition is a semi-solid or a solid.

11. A method for wetting a pigmented, transfer resistant film on the lips, comprising coating said transfer resistant film with a non-reactive wetting agent composition that serves to wet the transfer resistant film.

12. The method of claim 11 wherein the wetting agent composition is a liquid.

13. The method of claim 11 wherein the pigmented, transfer resistant film on the lips is a pigmented resinous composition.

14. The method of claim 13 wherein the pigmented resinous composition comprises a silicon resin or acrylic polymer in combination with one or more nonvolatile oils.

15. The method of claim 11 wherein the wetting agent composition comprises a high viscosity natural oil, hydrocarbon, surfactant, glyceryl ester, di- or polyhydric alcohol, water soluble polymer, polyalphaolefin, or perfluoropolyether.

16. The method of claim 15 wherein the wetting agent is a polyalphaolefin, perfluoropolyether, or mixtures thereof.

17. A multipack cosmetic composition comprising at least two separate receptacles in a single stock keeping unit, said first receptacle containing a pigmented transfer resistant composition, and said second receptacle containing a non-reactive wetting agent composition for said transfer resistant composition.

18. The multipack cosmetic composition of claim 17 wherein the two receptacles are in two separate cosmetic containers within a single stock keeping unit.

19. The multipack cosmetic composition of claim 18 wherein the two receptacles are in a single cosmetic container within a single stock keeping unit.

20. The multipack cosmetic composition of claim 18 which comprises an additional receptacle containing a remover composition for said transfer resistant composition and said wetting agent composition.
